(19) United States
(12) Patent Application Publication
(10) Pub. No.: US 2008/0125674 A1
(43) Pub. Date: May 29, 2008

(54) Catheter Guide Wire, in Particular for Cardiovascular interventions

(76) Inventors: Deniz Bilecen, Schopfheim (DE); Eugen Hofmann, Zurich (CH); Wladimir Ostheim-Dzerowycz, Riehen (CH)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(21) Appl. No.: 11/570,182

(22) PCT Filed: May 19, 2005
(86) PCT No.: PCT/EP05/05424

§ 371 Date: May 11, 2007

(30) Foreign Application Priority Data
Jun. 11, 2004 (DE) 10 2004 028 367.2

(51) Int. Cl. A61M 25/09

(52) U.S. Cl. 600/585

(57) ABSTRACT

The invention relates to a catheter guide wire comprising a PEEK core (e) that is provided with a coating (6). The core (4) extends in one piece from the proximal end to the distal end (3, 2) of the guide wire 81) and tapers towards the distal end (2). The coating (6) is provided with a contrast medium (7).

The present invention relates to a catheter guide wire, particularly for cardiovascular interventions, according to the features specified in the preamble of claim 1.

On the background of the present invention, it is to be noted that when catheters are placed in the course of cardiovascular interventions, for example, during percutaneous transluminal angioplasty (PTA), the catheter is positioned with the aid of a guide wire previously inserted into the body of the patient. X-ray fluoroscopy is common for monitoring the advance of the guide wire through the various blood vessels up to its target location, in addition, magnetic resonance fluoroscopy is currently in development for practical application.

In x-ray fluoroscopy, the guide wires are visible because of their material properties. In special cases, for example, for coronary applications, the guide wires have radiopaque markers, particularly on their distal ends, which stand out so they are clearly recognizable on an x-ray viewing device. However, because of the known disadvantageous effect on the human body of x-ray radiation and the contrast agent used, this x-ray visualization will probably lose significance to an increasing degree.

Magnetic resonance tomography (MRT in short in the following), in contrast, provides image information having more content which is stress free for the patient, if catheter guide wires are used which stand out clearly in a magnetic resonance viewing device.

In this regard, providing the guide wire with an elongate core made of a material having high specific electrical impedance, such as a glass fiber material, is known, for example, from WO 98/42268 A1.

Furthermore, using an elongate PEEK core as the core is known from the two publications US 2003/0120148 A1 and US 2003/0135114 A1, which relate to one another as the original and continuation applications. This core extends starting from the proximal end of the guide wire up to in front of its distal end and passes there into a distal end section, which comprises a nonmagnetic metal or alloy material. This is to be more flexible than the PEEK core of the proximal core section of the catheter guide wire.

The guide wire disclosed in the two above-mentioned publications is also provided on its distal end with a metallic coil, which is at least partially manufactured from nonmagnetic material, however, which provides a high contrast in an MRT device.

Overall, the previously known guide wire having its multipart core is implemented having an extremely complex construction. The present invention is thus based on the object of providing a magnetic-resonance-compatible guide wire, which is distinguished by a simple construction with high flexibility of the distal end and good recognizability in MRT devices.

This object is achieved by the features specified in the characterizing part of claim 1. Accordingly, the core of the guide wire is formed continuously from PEEK material in one piece from the proximal end up to the distal end and implemented having a tapering diameter to its distal end in the end area, i.e., essentially runs “to a point”. Furthermore, the guide wire is provided with a coating in which an MRT contrast agent is incorporated. Common MRT contrast agents are elements of the lanthanide group, such as gadolinium or dysprosium. The embedding of titanium particles as the MRT contrast agent in the coating is also conceivable. Iron powder is preferably integrated in the coating as the contrast agent.

Since the coating also has the object of ensuring good sliding properties for the guide wire upon insertion into a body vessel and for sliding over a catheter, this coating thus advantageously fulfills a typical double function.

It has been shown that a guide wire having a continuous PEEK core and the specified coating stands out clearly in MRT devices. This also relates to the tapering of the core provided to increase the flexibility of the distal end at this end. Overall, the catheter guide wire according to the present invention has good MRT compatibility, since it displays few artifacts in MRT viewing devices in any case. In particular, there is no heating of the guide wire, as is the case if x-ray-compatible guide wires are used in MRT viewing devices.

Because of the tapering of the core toward the distal end, the required flexibility of the distal end for advancing the guide wire is ensured even around tighter bends, through corresponding filigree branches and passages in the cardiovascular system.

Preferred embodiments of the present invention as well as further features, details, and advantages may be inferred from the subclaims and the following description, in which an exemplary embodiment of a catheter guide wire is explained in greater detail on the basis of the attached drawing.

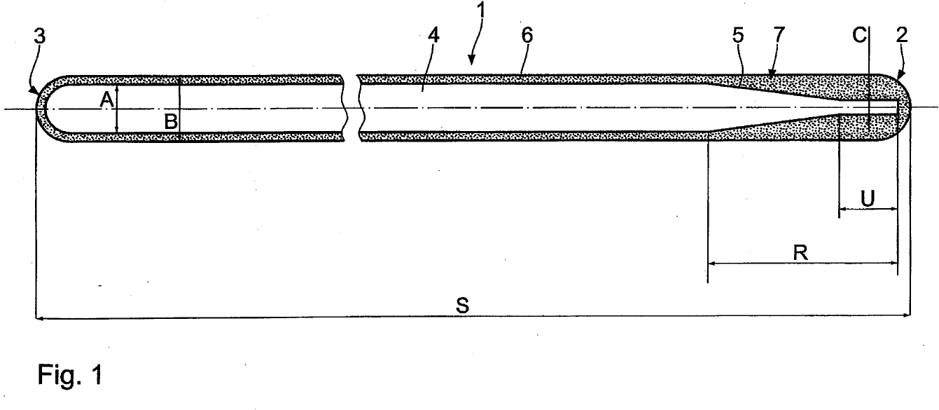

FIG. 1 shows a schematic longitudinal axial section of a catheter guide wire.

The catheter guide wire identified as a whole in the drawing by 1—the name “wire” is typical in medical technology, in spite of the fact that the object does not comprise metal—has a distal end 2 to be inserted into the body of the patient and a proximal end 3, which remains outside the body for handling the guide wire 1. The supporting part of the guide wire 1 is a core 4, which is implemented as an elongate polyether ether ketone (PEEK) monofilament. Up to a short end area R of approximately 100 mm before the distal end 2, the core 4 has a constant diameter A of approximately 0.7 mm. A conical taper 5 to the distal end 2 is provided in the end area R, in the course of which the diameter of the core 4 is reduced further to value C of 0.25 mm, for example. In a short tip section U of 20 mm length, for example, the core 4 has this constant external diameter C. The overall length S of the catheter guide wire is to be tailored to the particular patient and may be 1500 mm, for example.

The guide wire 1 is provided over the entire surface of its core 4 with a single-component or multicomponent coating 6, which may have a thickness of approximately 0.07 mm in the area of the constant diameter A. The total external diameter B of the guide wire is thus 0.85 mm and remains constant over the entire length, i.e., even in the distal end area R. Correspondingly, as may be seen well in the drawing, the coating thickness increases in the distal end area R in accordance with the diameter reduction of the core 4.

The materials usable for the possibly multilayered coating 6 must primarily be friction-reducing in such a way that the insertion of the guide wire into body vessels and sliding the catheter over the guide wire may occur as smoothly as possible. Relevant coating materials are polyurethanes and polyamides, which may be used as an inner layer of the coating. Hydrophilic silicon or polysiloxane coatings come into consideration for friction reduction for the outer layer. In the design of the material of the coating 6, it is to be ensured that the core 4 has a higher bending strength than the coating, so that the distal end area R experiences an increase of its flexibility because of the diameter reduction of the core.

As is indicated highly schematically and only in a partial area by dotting in the drawing, the coating 6 is admixed over its entire volume with iron powder 7, which has a grain size of less than 10 μm. Its proportional amount in relation to the coating is approximately 1 to 2 weight-percent. The iron powder 7 in the coating 6 results in improved recognizability of the guide wire both in x-ray radiation devices and also MRT devices, since it stands out well as an x-ray contrast agent and, in addition, generates a “quenching effect”. Although this is an interference effect per se, the guide wire may thus be recognized well by MRT detection. Due to the thickness increase of the coating 6 at the distal end 2 and the higher quantity of iron powder 7 in this wire area connected thereto, this end area R is also recognizable especially well.

It is to be noted on the production procedure for the catheter guide wire that the PEEK core is extruded, subsequently straightened by orientation, and finally ground down in the distal end area R.

1. A catheter guide wire, particularly suitable for use in cardiovascular interventions, comprising:
a) a distal end;
b) a proximal end;
c) an elongate polyether ether ketone core being continuous in one piece from the proximal end up to the distal end of the guide wire, the core tapering in the end area from a proximal diameter at its proximal end to a distal diameter at its distal end; and
d) a coating on the core admixed with a contrast agent,

2. The catheter guide wire of claim 1, wherein the coating is admixed with a contrast agent which is detectable using both x-ray radiation and magnetic resonance.

3. The catheter guide wire of claim 2, wherein the contrast agent comprises iron powder.

4. The catheter guide wire of claim 3, wherein the iron powder comprises about 1 to 2 weight-percent in relation to the coating.

5. The catheter guide wire of claim 1, wherein the core has a higher bending strength than the coating.

6. The catheter guide wire of claim 1, wherein the core comprises an extruded polyether ether ketone monofilament.

7. The catheter guide wire of claim 1, wherein the coating thickness is increased in the end area such that the external diameters of the guide wire is substantially constant over its length.

8. The catheter guide wire of claim 1, wherein the ratio of the proximal diameter of the core to the distal diameter is approximately 2:1 to 5:1.

9. The catheter guide wire of claim 1, wherein the coatings includes a friction-reducing polymer layer.

10. The catheter guide wire of claim 6, wherein the coating includes a hydrophilic outer layer.

11. The catheter guide wire of claim 3, wherein the contrast agent has a grain size of less than 10 μm.

12. The catheter guide wire of claim 1, wherein the ratio of the proximal diameter of the core to the distal diameter is approximately 3:1.
